(19) United States
(12) Patent Application Publication
(10) Pub. No.: US 2019/0380848 A1
(43) Pub. Date: Dec. 19, 2019

(54) Adjustment Mechanism for Prosthetic Socket

(71) Applicant: LIM Innovations, Inc., San Francisco, CA (US)

(72) Inventors: Garrett Ray Hurley, San Francisco, CA (US); Loren Maxwell Brock, Sebastopol, CA (US)

(73) Assignee: LIM Innovations, Inc., San Francisco, CA (US)

(21) Appl. No.: 16/440,698

(22) Filed: Jun. 13, 2019

Related U.S. Application Data
(60) Provisional application No. 62/736,945, filed on Sep. 26, 2018.
(60) Provisional application No. 62/684,813, filed on Jun. 14, 2018.

(51) Int. Cl. A61F 2/80; A61F 2/68; A61F 2/66

(52) U.S. Cl. CPC A61F 2/80; A61F 2/68; A61F 2002/608; A61F 2220/0025; A61F 2/66

(57) ABSTRACT

A prosthesis system for connection to a user's residual limb includes a socket defining a cavity to receive the residual limb. The system includes a cable laced about the socket. The cable has at least one portion that extends below a distal end of the socket. The system includes an adjustment mechanism coupled to the socket and disposed below the distal end of the socket. The adjustment mechanism is coupled to the at least one portion of the cable extending below the distal end of the socket. The adjustment mechanism is configured to adjust the tension in the cable for adjusting the fit between the socket and the residual limb. Also, the system includes a prosthetic extremity coupled to the socket and extending below the socket.

### CROSS REFERENCE TO RELATED APPLICATIONS

This application claims benefit to US Provisional App. No. 62/736,945, filed Sep. 26, 2018, and US Provisional App. No. 62/684,813, filed Jun. 14, 2018, both of which are hereby incorporated by reference herein in their entireties.

### BACKGROUND

### 1. Field

The present disclosure relates to prosthetic devices and related systems and methods.

### 2. State of the Art

The use of prostheses by transtibial amputees is generally well known. Transtibial prostheses can include a socket, a pylon, and a foot-ankle system. A variety of sockets, pylons, and foot-ankle systems are available, which can be combined in any suitable manner to produce a transtibial prosthesis that is tailored to meet the individual needs of different transtibial amputees. The socket generally acts as the structural component of the prosthesis that contains the residual limb and provides connection to the other components. The socket is instrumental in transferring the weight of a transtibial amputee to the ground by the way of the prosthesis. In turn, the pylon transfers vertical loads (e.g., at least a portion of the weight of the amputee) from the socket to the foot-ankle system, which interacts with the ground. If the socket does not fit and operate properly, utility of the distal components can be severely compromised.

Whether the prosthesis is a transtibial or transfemoral prosthesis, or even an upper limb prosthesis (such as for upper or lower arm amputees), the interface between the prosthesis and the person's residual limb is of great importance. The socket portion of the prosthesis typically defines the primary interface between the prosthesis and the residual limb. Several factors can be weighed in the design of a socket, including whether the socket satisfactory transmits the desired load, provides satisfactory stability, provides efficient control for mobility, is easily fitted, and/or is comfortable.

The residual limb typically changes size not only over months or years as the amputee's body ages or recovers from the initial amputation, but also on daily basis, and even throughout a given day. The daily or short-term fluctuations in residual limb size can be a result of water retention or loss. The more active an amputee is throughout the day, the greater the water loss in the residual limb may be. This change in size can have an effect on the fit between the residual limb and the prosthetic socket. Amputees often account for such a reduction in limb size by adding a sock to the limb. Adding the sock often requires a person to remove an article of clothing, remove and then replace the prosthesis, and then put back on the removed article of clothing. This process not only can be time consuming, but it can also require a certain amount of privacy. In many instances, a number of socks (e.g., 3, 5, 10, or more) may be added to the residual limb throughout the day in order to maintain adequate fit between the residual limb and socket for the amputee to avoid the pain and discomfort that can result from an improper fit.

Various prosthetic devices that are adjustable relative to a residual limb of an amputee have been proposed. An example of one such device is described in U.S. Pat. No. 9,956,094 (Mahon). Specifically, Mahon describes one embodiment of a prosthetic device that has proximal tensioning lines, distal and proximal guide members, and distal and proximal adjustment mechanisms. The adjustment mechanisms protrude radially outward from the socket a predetermined distance. Such a protrusion can make it difficult to cover when wearing pants and can create an unsightly and unnaturally appearing bulge under such clothing.

Another example of a prosthetic device that is adjustable relative to a residual limb of an amputee is described in U.S. Pat. No. 8,978,224 (Hurley et al.). Hurley describes a proximal brim member that has encircling bands with internal tensioning cables that run through the inside of the encircling bands. The bands can be tightened or loosened by a rotary tensioning mechanism on the outside of the bands.

### SUMMARY

According to one aspect, further details of which are described below, a prosthesis system for connection to a user's residual limb includes a socket having a plurality of structural struts, a distal base supporting the struts and forming a distal end of the socket, and an interface received within the struts and vertically above the base. The interface defines a shape-conformed cavity adapted to receive the residual limb. The distal end of the base includes a mount at which to receive a pylon to which is coupled a modular prosthetic extremity, such as an ankle and foot system. Also, the system includes a cable laced about the socket. The cable has at least one portion that extends below the distal base of the socket. The system also includes an adjustment mechanism coupled relative to the socket and, in embodiments, disposed below the distal end of the socket. In an embodiment, the adjustment mechanism is coupled to the pylon. The adjustment mechanism is coupled to the at least one portion of the cable extending below the distal end of the socket. The adjustment mechanism is configured to adjust the tension in the cable for adjusting the fit between the socket and the residual limb. Also, the system includes a prosthetic extremity coupled to the socket and extending below the socket.

The system may include a mounting bracket that attaches the adjustment mechanism to an socket between the distal end of the socket and the prosthetic extremity. The bracket may have a first flange that defines at least one hole. The at least one hole is preferably elongated. Also, the at least one hole is configured to align with a corresponding fastener between the distal end of the socket and the prosthetic extremity. The bracket may have a second flange extending at an angle with respect to first flange, and the adjustment mechanism may be mounted to the second flange. The system may optionally include a strap extending from the bracket or the adjustment mechanism to the prosthetic extremity.

In embodiments, at least one of the interface and the struts are provided pathways through which the cable is routed. In embodiments, the system may include cable guides coupled to the socket. The cable guides are configured to guide the cable about at least portions of the socket. In embodiments, the adjustment mechanism is configured to tighten and loosen the cable about the socket. The adjustment mechanism may be a rotary winding mechanism. The adjustment mechanism may be coupled to a lever of a latch and moves with the latch between an open configuration and a closed configuration of the latch.

### BRIEF DESCRIPTION OF THE DRAWINGS

FIG. 1A is an isometric view of an adjustable prosthetic system in accordance with an aspect of the disclosure, viewed from a rear and right side.

FIG. 1B is shows the system of FIG. 1A with a shoe.

FIG. 1C is an isometric view of the adjustable prosthetic system of FIG. 1A viewed from a front and right side.

FIG. 1D shows a rear view of the system of FIG. 1A with cables and cable housings attached.

FIG. 1E shows a right side view of the system of FIG. 1D.

FIG. 1F shows a front view of the system of FIG. 1D with cables and cable housings attached.

FIG. 1G shows a left side view of the system of FIG. 1D.

FIG. 2A is an exploded assembly view of the adjustable prosthetic system of FIG. 1A.

FIG. 2B shows a bracket of the adjustable prosthetic system of FIG. 1A.

FIG. 2C shows a side elevation view of the bracket shown in FIG. 2B.

FIG. 2D is an isometric view of a receiver or base of an adjustment mechanism of the prosthetic system of FIG. 1A, viewed from a front and right side.

FIG. 2E is a rear view of the receiver shown in FIG. 2D.

FIG. 2F shows the components of the assembly in FIG. 2A in an assembled configuration.

FIGS. 3A to 3C show details of anterior cable guides of the prosthetic system of FIG. 1A.

FIGS. 4A and 4B show details of a pressure adjustment using the prosthetic system of FIG. 1A.

FIG. 5A shows another embodiment of an adjustable prosthetic system in accordance with the disclosure, where the system is viewed from a posterior side.

FIG. 5B shows the system of FIG. 5A viewed from anterior and superior sides.

FIG. 5C shows the system of FIG. 5A viewed from the anterior side.

FIG. 5D shows a detailed view of a portion of the system shown in FIG. 5A viewed from a lateral side.

FIG. 5E shows the system of FIG. 5A viewed form the anterior and lateral sides.

FIG. 5F shows the system of FIG. 5A viewed form the anterior and medial sides.

FIG. 6A shows details of an alternative embodiment of an adjustment mechanism for the system of FIG. 5A.

FIG. 6B shows details of a bracket used with the adjustment mechanism shown in FIG. 6A.

FIG. 6C shows the adjustment mechanism of FIG. 6A in an intermediate configuration between a closed and open configuration.

FIG. 6D shows the adjustment mechanism of FIG. 6A in an open configuration.

FIG. 7A shows a front view of another embodiment of an adjustable prosthetic system in which an adjustment mechanism is disposed in spaced relation from a pylon flange.

FIG. 7B shows a side view of a portion of the system in FIG. 7A with an adjustment mechanism disposed adjacent to a pylon flange.

FIG. 8 shows a top and front view of an alternate mounting arrangement for an adjustment mechanism.

FIGS. 9A to 9C show another embodiment of a mounting arrangement for an adjustment mechanism.

FIGS. 10A to 10C show another embodiment of a mounting arrangement for an adjustment mechanism.

FIG. 11 shows another embodiment of an adjustable prosthetic system in accordance with an aspect of the disclosure.

FIG. 12A shows rear view of the adjustable prosthetic system with a pylon connected and misaligned with the axis of the socket.

FIG. 12B shows an exploded view of a portion of the system in FIG. 12A.

FIG. 12C shows a rear view of the system in FIG. 12A.

FIG. 12D shows a right side of the system in FIG. 12A.

FIG. 12E shows a left side of the system in FIG. 12A.

FIG. 12F shows a top side of the system in FIG. 12A.

FIG. 13A shows a side view of a prosthetic system and another mounting arrangement for an adjustment mechanism in accordance with an aspect of the disclosure mounted to a pylon.

FIGS. 13B and 13C show details of the mounting arrangement of FIG. 13A.

FIG. 13D shows a side view of the mounting arrangement of FIG. 13A mounted to a foot-ankle structure.

FIG. 14A shows a side view of a prosthetic system with the mounting arrangement of FIG. 13A along with a cable guide in accordance with an aspect of the disclosure.

FIG. 14B shows the mounting arrangement of FIG. 14A and a kit that comprises the cable guide of FIG. 14A.

FIG. 14C is a rear view of a portion of the system of FIG. 14A.

FIG. 14D is a left side view of the system of FIG. 14A with the socket moved further in a posterior direction relative to the pylon.

FIG. 15A shows a side view of a prosthetic system with another embodiment of a mounting arrangement in accordance with an aspect of the disclosure.

FIGS. 15B and 15C show details of the mounting arrangement of FIG. 15A.

### DETAILED DESCRIPTION

For the sake of convenience, much of the following disclosure is directed to prosthetic devices that are configured for use with a residual portion of an amputated leg, such as a leg that has undergone a transfemoral (i.e., above-knee) or transtibial (i.e., below-knee) amputation. It should be appreciated that the disclosure is also applicable to other prostheses, such as those configured for use with the residual limb of an amputated arm (e.g., after an above-elbow or below-elbow amputation).

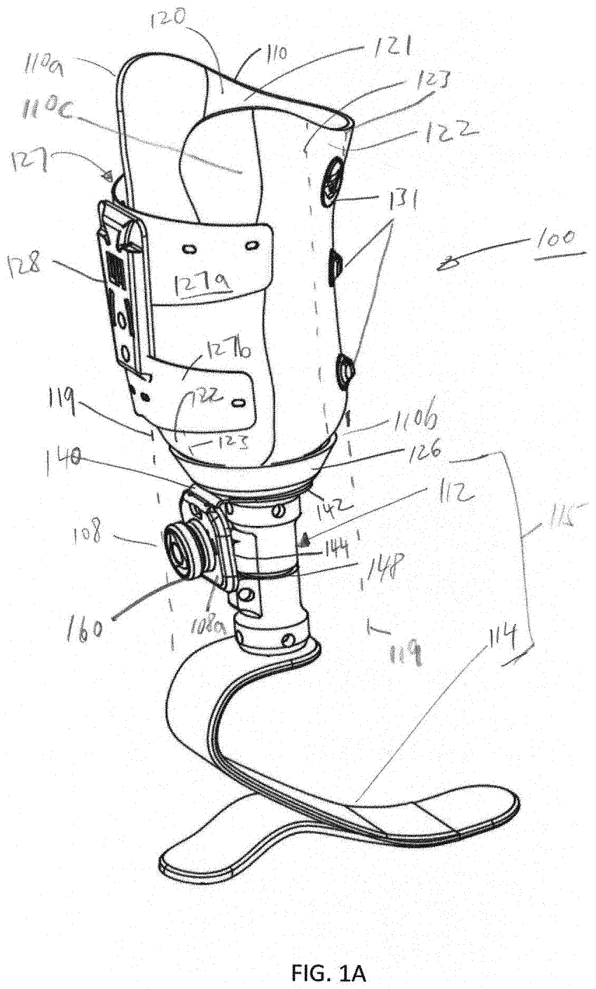

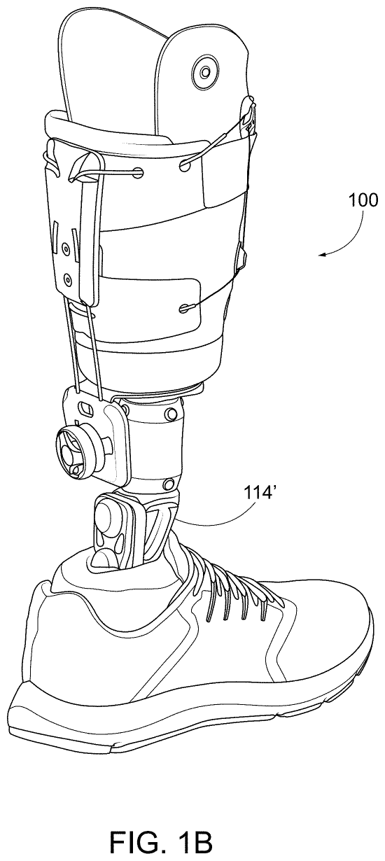

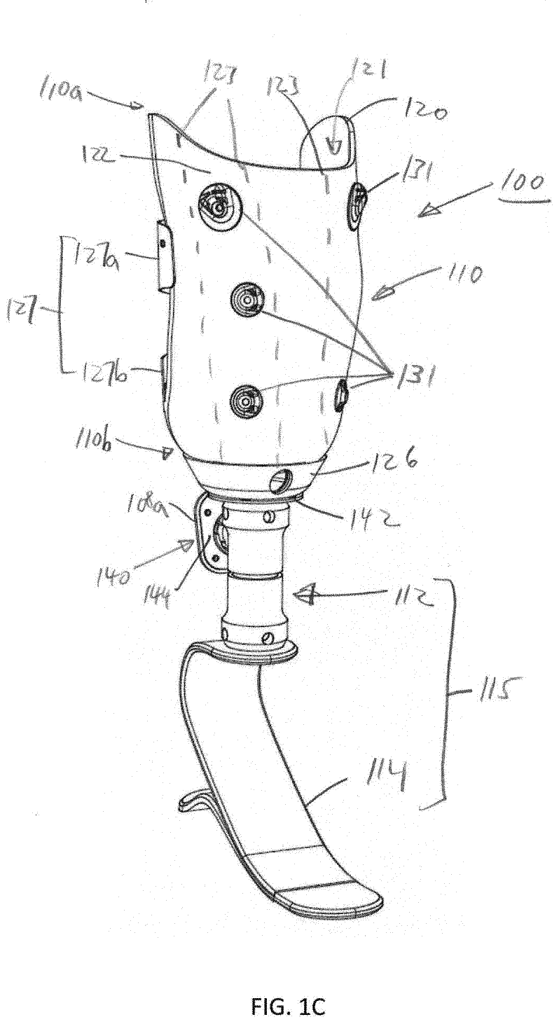

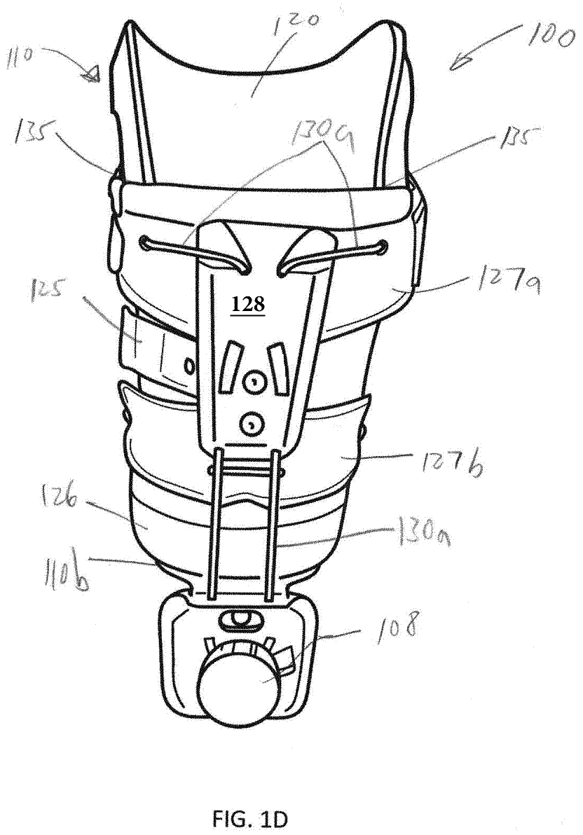

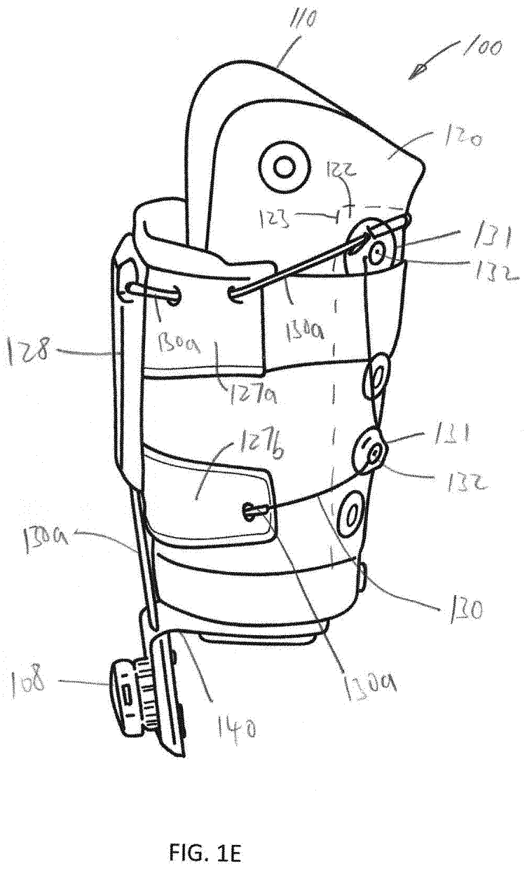

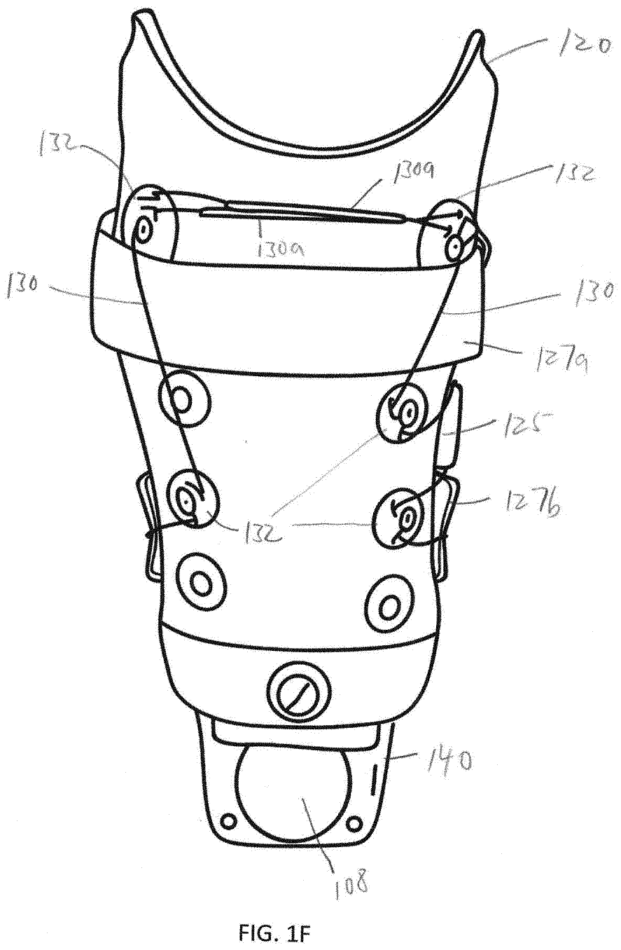

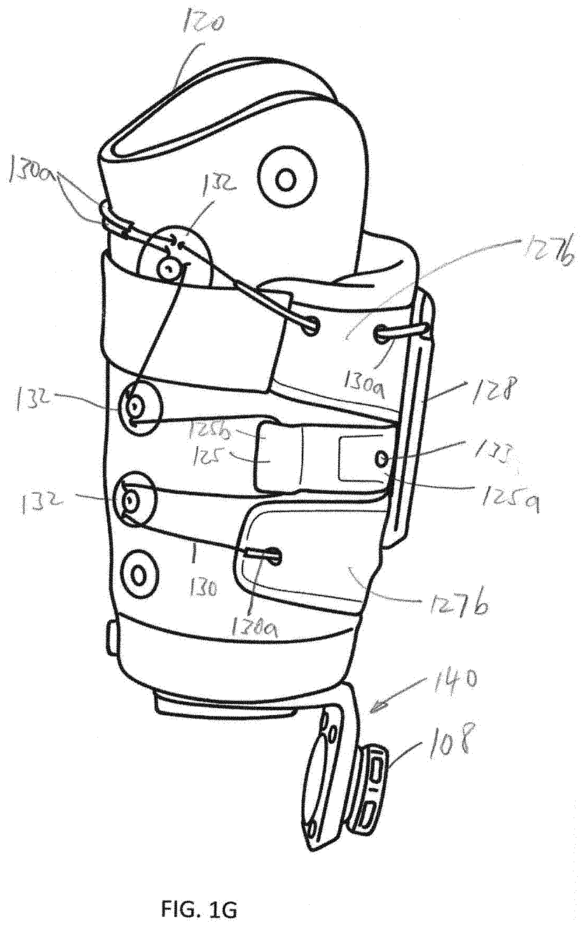

FIG. 1A illustrates an embodiment of an adjustable prosthetic system 100 that includes a prosthetic socket 110, a control cable (also referred to herein as a tension element) 130 (FIGS. 1E-1G), an adjustment mechanism 108, and a modular prosthetic extremity 115. In the illustrated embodiment, the system 100 is configured as a substitute for a portion of a leg of an amputee. The adjustment mechanism 108 and cable 130 may be, by way of example, components of the BOA Fit System manufactured by BOA Technology Inc. of Denver, Colo.

As described further below, the socket 110 includes an interface 120 that defines a cavity 121 that is configured to receive a residual limb of a leg (not shown) therein. The socket 110 serves as a connection between the residual limb and the prosthetic extremity 115, which includes a support or pylon 112 and an ankle-foot structure 114. Any suitable arrangement of the prosthetic extremity 115 is possible. Another ankle-foot structure 114a is shown in FIG. 1B that is connected to a shoe.

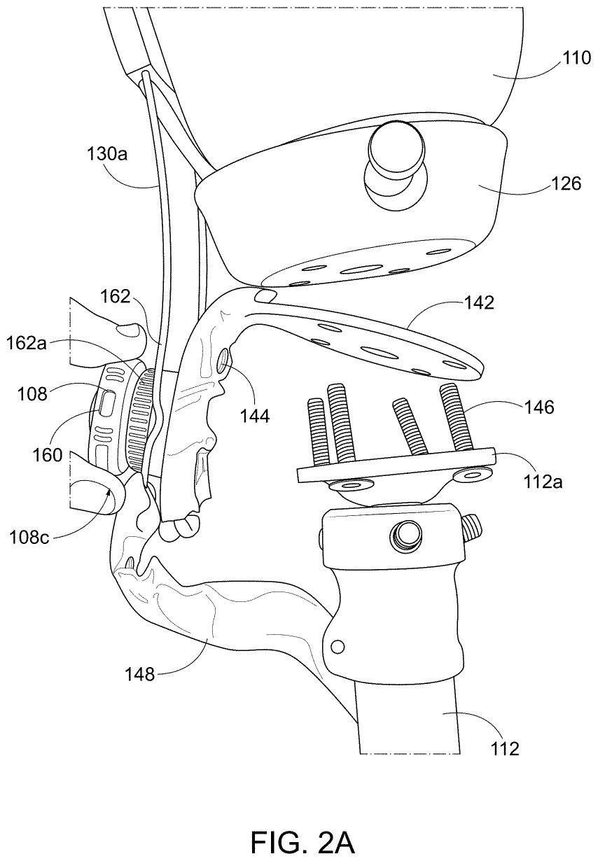

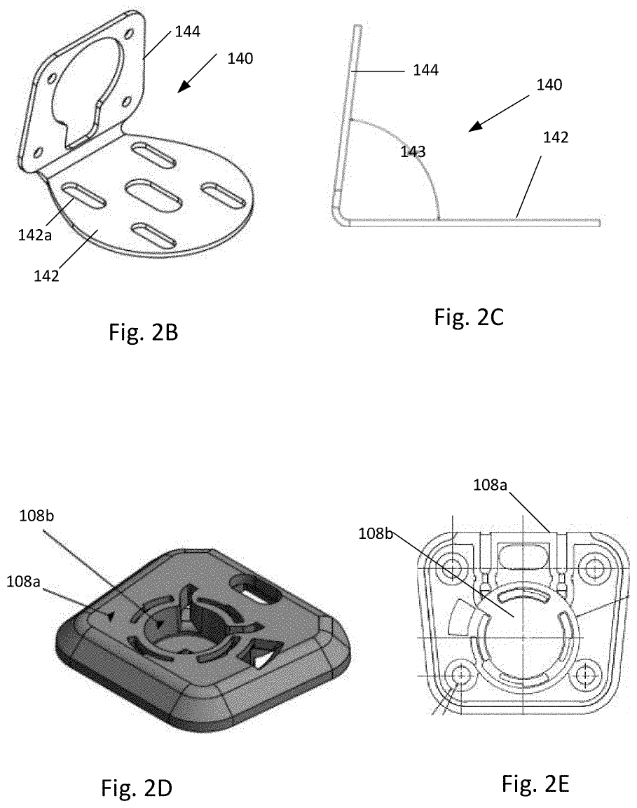

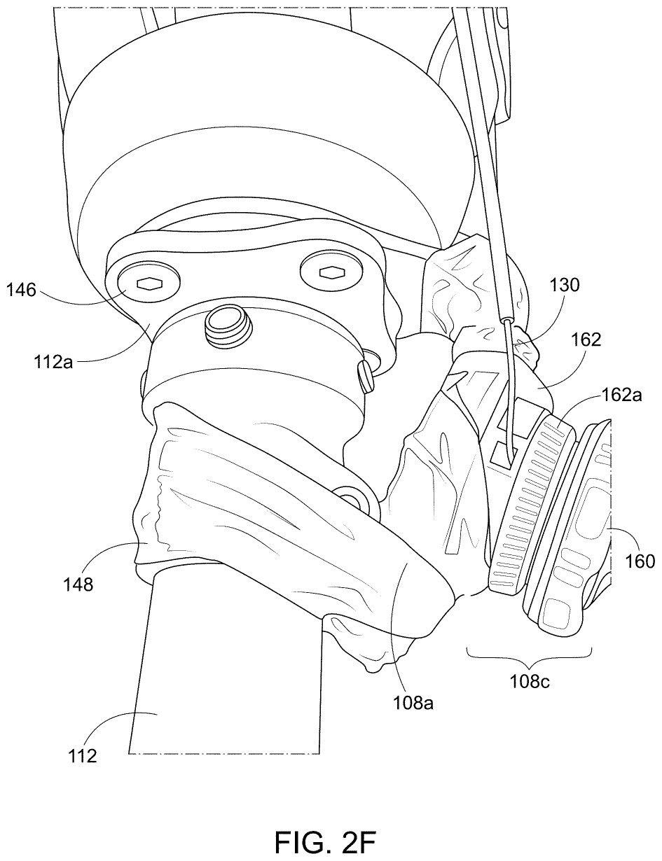

As shown in FIG. 1A, the socket 110 extends from a proximal end 110a to a distal end 110b. The adjustment mechanism 108 is mounted to a bracket 140 that disposes the adjustment mechanism 108 below the distal end 110b of the socket 110 and is supported between the socket 110 and the prosthetic extremity 115 and may optionally be supported by the pylon 112 directly as well, such as with a strap 148 (FIG. 2A) or clamp. While the adjustment mechanism 108 is shown in FIG. 1A as being disposed on a posterior side, the adjustment mechanism 108 may be disposed at other circumferential positions relative to axis A-A, such as on a medial, anterior, or lateral side.

In the embodiment shown in FIG. 1A, the adjustment mechanism 108 does not extend radially beyond the projected profile of the outer surface of the socket 110, as represented by broken lines 119 in FIG. 1A. Such a configuration can allow the user to wear pants over the system 100 without having an unsightly bulge caused by the adjustment mechanism 108 extending outward from the surface of the socket 110.

The socket 110 has a base 126 at its distal end 110b to which the pylon 112 is coupled. The socket 110 includes a plurality of structural struts 122, which may be internal to the socket 110 structure (i.e., may be in an inner layer or otherwise covered) and shown in broken lines 123 along the socket 110. The socket 110 also includes an interface 120 retained at the interior of the socket 110 and along the proximal side of the distal base 126. The struts 122 generally extend in a proximal-distal (i.e., vertical) direction and are coupled at their distal ends to the base 126 so that the struts 122 can flex radially about the base 126. The interface 120 may overlap upper ends of the struts 122 (which may be spaced distally from the proximal end 110a of the socket 110) and/or the upper end of an outer layer 110c (FIG. 1A) of the socket 110 to retain the struts 122 to the interface 120 and to space them circumferentially relative to one another. Also, the overlap of the interface 120 over the struts 122 may render the proximal ends of the struts 122 atraumatic to the residual limb. The interface 120 can be configured to provide a high amount of surface contact with the residual limb to achieve a close fit therewith, which can correlate with the high amount of comfort for the user. Thus, in one embodiment, the socket 110 may be formed as a layered structure with an inner layer comprised of the interface 120, a middle or intermediate layer comprised of the struts 122, and an outer layer 110c, which may be formed of plastic or carbon fiber, for example.

The interface 120 defines the cavity 121 into which the residual limb (along with a custom prosthetic liner e.g., a roll-on liner, not shown), can be received. The interface 120 is also custom made to complementarily fit the contours of the custom prosthetic liner worn over the residual limb. The interface 120 may be formed of a first material (e.g., plastic such as polypropylene or a fiber-composite matrix) that is softer and less rigid than a second material (e.g., epoxy filled carbon fiber or other fiber-composite matrix) comprising the struts 122. Nonetheless, the interface 120 may still be considered substantially rigid so as to maintain its shape or form when forces are applied thereto, whether from the residual limb when it is positioned therein or from compressive forces at an exterior thereof. The term “substantially rigid” is sufficiently broad to cover arrangements where the interface 120 is sufficiently rigid, solid, or firm so as to undergo no change in shape or configuration due to stresses applied thereto by the residual limb under normal use (i.e., solid), as well as arrangements where the interface 120 is very rigid, solid, or firm, but is resilient and may undergo slight, non-permanent deformations due to the standard stresses of use (i.e., flexibly firm).

As shown in FIG. 1A, a posterior peninsula 128 and a posterior firmwear 127 are coupled to the socket 110. The peninsula 128 is preferably secured to one of the struts 122 that extends in vertical alignment with the peninsula 128 at a posterior of the socket 110. The posterior firmwear 127 includes a proximal firmwear 127a and a distal firmwear 127b, which both extend from the peninsular 128 generally in a circumferential direction. As shown in FIGS. 1D to 1G, the proximal firmwear 127a extends fully about the interface 120. The proximal firmwear 127a has a pad 129 on an inner side of the proximal firmwear 127a (with respect to the side facing the cavity 121). The pad 129 is configured to engage the residual limb in the cavity 121.

Also, a pressure management strap 125 (FIG. 1D) extends circumferentially from one lateral side of the peninsula 128. The pressure management strap 125 has a first adjustable end 125a that is connected to the peninsula 128 with a set screw 133 and has a second end 125b that has an opening to route cable 130, as shown in FIG. 1G. When the set screw 133 is loosened, the adjustable end 125a can be moved laterally to adjust the circumferential position of the second end 125b, which can facilitate pressure adjustment, further details of which are described hereinbelow. The peninsula 128 and the firmwear 127 are configured to guide cable 130 (FIGS. 1D to 1G) about the socket 110. Due to the connection of the struts 122 to the base 126, the entire peninsula 128 and the firmwear 127 can move radially inward and outwardly with respect to the cavity 121, further details of which will be described below.

As shown in FIGS. 1E-1G, anterior cable guides 131 are also connected to the socket 110. The cable guides 131 may connect to the underlying struts 122 of the interface 120, such as with a fastener (e.g., screw) 132.

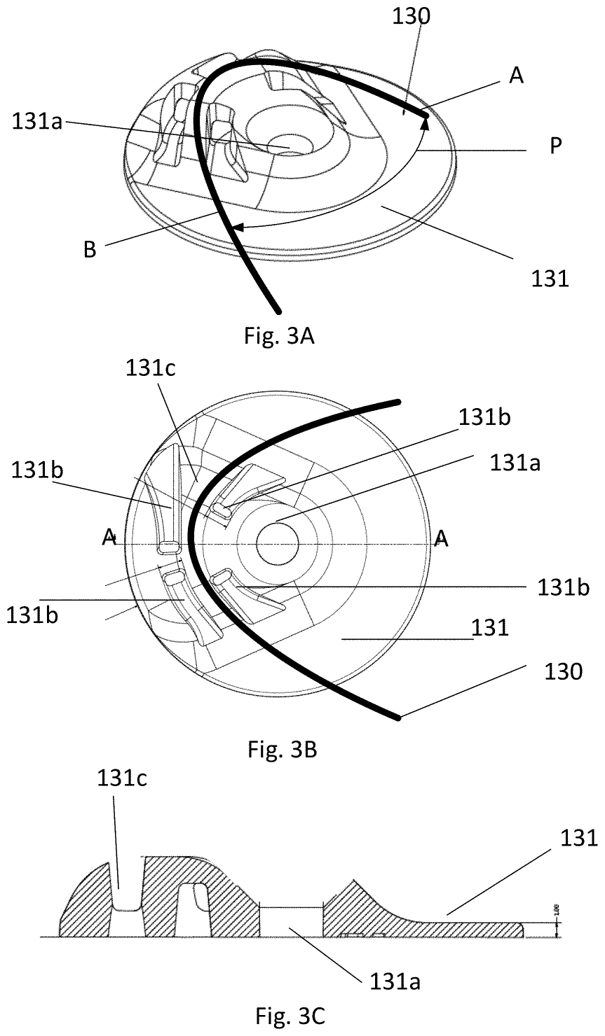

With momentary reference to FIGS. 3A to 3B, the cable guides 131 are configured to route two segments (A and B) of the cable 130 at an angle of pull (P) therebetween, as shown in FIG. 3A. The cable guides 131 are configured to be adjusted relative to the interface 120 by turning the cable guide 131 about the fastener 132 such that the orientation of the cable guide 131 evenly divides the angle of pull about the fastener 132 and relative to an axis A-A (FIG. 3B) extending through the cable guide 131.

Turning back to FIGS. 1D to 1G, the cable 130 is routed through the peninsula 128, the firmwear 127, the cable guides 131, and the pressure management strap 125, thereby defining a cable routing path around the socket 110. Such routing may be similar to shoelace lacings in footwear, such as ski boots, or may have another routing path suitable for applying the desired tension force from the cable 130 to the socket 110.

Along some portions of the cable routing path, the cable 130 may be covered by respective cable housings 130a. In FIG. 1D, cable housings 130a extend from the adjustment mechanism 108 to the bottom of peninsula 128 and between the top of the peninsula 128 and the proximal firmwear 127a. Also, as shown in FIGS. 1E and 1G, cable housings 130a extend around a proximal anterior portion of the interface above the proximal firmwear 127a, and extend from the distal firmwear 127b.

The tightening of the cable 130 preferably effects circumferential compression of the socket 110 and also the interface 120 about the residual limb; loosening of the cable 130 effects loosening of the socket 110 and the interface 120. The cable 130 is routed at least partially about the socket 110 and down to the adjustment mechanism 108, which, in FIG. 1D to 1G, is disposed below the distal end 110b of the socket 110. Portions (e.g., terminal portions) of the cable 130 are routed below the base 126 of the socket 110 to the adjustment mechanism 108, which is shown as a rotary ratcheting mechanism, such as a mechanism manufactured by BOA Technology Inc. of Denver, Colo. and like one described in U.S. Pat. No. 7,992,261, the entire contents of which are incorporated herein by reference. The adjustment mechanism 108 is configured to wind the cable 130 to shorten the length of the cable 130 laced about the socket 110, which effects tensioning of the cable about the interface 120.

In the case of the rotary ratcheting mechanism 108, the mechanism has a winding spool for winding the cable 130 to tighten the cable 130 and the mechanism may have a configuration that permits the cable 130 to be unwound from such spool to loosen the cable 130. Further details of the adjustment mechanism 108 and its functions are described below.

The struts 122, the interface 120, and the firmwear 127 are configured to compress radially inwardly and outwardly relative to the cavity 121 based on the tension in the cable 130. When the cable 130 is tensioned by shortening the length of the cable 130 about the socket 110 (during an adjustment procedure) the cable 130 can slide relative to the cable housings 130, the cable guides 132 and the pressure management strap 125 such that the circumference of the socket 110, and thus, the interface 120 can be effectively reduced to make more snug the fit between the interface 120 and the residual limb in the cavity 121. The discontinuities between the cable housings 130a allow for the circumferential change of the socket 110, and, consequently, the interface 120.

As shown in greater detail in FIG. 1D, the peninsula 128 routes the cable 130 through the proximal firmwear 127a in a manner to direct a force vector along the cable 130 to compress the interface 120 and the proximal firmwear 127a against the residual limb when present in the cavity 121. Specifically, as shown in FIG. 1D, the pad 129 contacts the interface 120 at two circumferentially spaced positions 135. When the pad 129 and the peninsula 128 are moved radially inwardly due to tightening of the cable 130 as discussed above, the interference between the pad 129 and the interface 120 at locations 135 causes the curvature of the pad 129 to flatten. This effect can be more pronounced depending on the difference in compliance between the materials of the interface 120 and the pad 129. For example, where the interface 120 is much more rigid than the pad 129 the pad 129 will bend and flatten much more readily than if the pad 129 were relatively stiffer. The ability of the pad 129 to flatten is different from what is described in U.S. Pat. No. 9,956,094 (Mahon). Mahon describes embodiments where separate tensioning pads are arranged to apply compressive force against a user's limb when tension is applied to tensioning lines. In Mahon, the tensioning pads move radially in and out of a window cut in an outer layer of a socket and apply pressure to an inner layer of the socket, which is akin to the interface 120 described herein. However, as shown in FIG. 30A of Mahon, for example, the pad (e.g. pad 1318c) does not engage with the outer layer of the socket and can actually move completely through (inwardly) the opening of the outer layer.

The adjustment mechanism 108 can allow the user to set the tension of the cable 130 incrementally by rotating the adjustment mechanism 108. Such adjustment can be considered an analog adjustment (rather than discrete or on/off adjustment) because it allows the user to “dial in” or otherwise make fine adjustments to the tension in the cable 130 (and thereby fine adjustments to the compression of the residual limb of the user within the socket 110) without having to completely release the tension during each adjustment. This allows the user to continuously make fine adjustments to the tension while wearing the system 100 throughout their daily activities. Also, the adjustment mechanism 108 can include a release mechanism to release some or all of the tension in the cable 130 to allow the interface 120 and struts 122 to radially expand to loosen the interface 120 on the residual limb.

The angle at which the cable 130 extends tangentially from a knob 160 of the adjustment mechanism 108 may affect the vector direction of the force applied from the cable 130 to the interface 120 and, consequently, the amount of torque needed to tighten the cable 130 about the interface 120 to obtain a certain amount of compression of the interface 120 onto the residual limb of the user. For example, in the embodiment shown in FIG. 1A, the cable 130 extends at a right angle with respect to a radial line extending from the center of the knob 160 to the point of tangency with the cable 130. Thus, when the cable 130 is tightened, the force vector is substantially vertical. The vertical force can be redirected partially horizontally by the peninsula 128 so that there is compressive force to compress the interface 120 radially inwardly and move the firmwear 127 (and the pad 129 connected thereto) radially inwardly.

FIGS. 2A to 2E illustrate details of a connection between the socket 110, the bracket 140, and the pylon 112. The bracket 140 is shown in greater detail in FIGS. 2B and 2C and shows a first flange 142 and a second flange 144 that extends downward and at a non-zero angle 143 (FIG. 2C) from the first mounting flange 142. The angle 143 may be less than, greater than, or equal to 90 degrees. Also, shown in FIGS. 2A and 2F is an optional strap 148, which connects a free end of the second flange 144 to the pylon 112 and can thus provide additional support to the bracket 140 and the mounted adjustment mechanism 108, especially when the cable 130 is under tension.

The first mounting flange 142 defines a plurality of holes 142a, at least one of which may be elongated as shown in FIG. 2B. In an embodiment, five holes 142a are provided in the first mounting flange 142, including a central hole and four surrounding holes positioned at corners of a virtual rectangle or square. The holes 142a are arranged in a pattern such that some or all of the holes 142a align with holes formed in the bottom of base 126 to receive fasteners 146 (FIG. 2A) therethrough, to connect a flange 112a of the pylon 112 to the base 126 and interconnect bracket 140 therebetween. Specifically, when fully assembled, as shown in FIG. 2F, the first mounting flange 142 is interposed between the flange 112a of the pylon 112 and the base 126, and is retained therebetween with the fasteners 146. In other embodiments, the holes 142a may have other shapes, such as shaped holes or slots circumferentially spaced from one another about a central hole, which may be circular or elongated.

The holes 142a are elongated so that the first flange 142 can be disposed slightly off center or biased from the corresponding holes in the bottom of base 126 to clear any other components that may be at the proximal end of the pylon 112. For example, in the case of FIG. 2A, if the pylon flange 112a was wider, the elongated holes 142a could permit the flange 140 to be translated slightly in the posterior direction relative to the base 126 to provide clearance for the larger flange 112a. If the holes 142a are circumferentially spaced slots, the first flange 142 can be translated in rotation to provide necessary clearance for otherwise interfering structure on the pylon.

In an embodiment, the second flange 144 extends downward with respect to the first flange 142. The second flange 144 is connected (e.g., with screws) to a receiver or base 108a of the adjustment mechanism 108, as shown in FIG. 1A. As shown in greater detail in FIGS. 2D and 2E, the receiver 108a defines a cavity 108b, which is configured to receive a winding portion 108c (FIGS. 2A, 2F) of the adjustment mechanism 108, further details of which are described below.

The angle 143 (FIG. 2C) between the second flange 144 and the first flange 142 may be preformed. Multiple brackets may be available with different angles between the respective second and first flanges 144 and 142. The various angles 143 will dispose the second flange 144 and the adjustment mechanism 108 at various positions relative to the pylon 112 when the bracket 140 is connected. Such multiple brackets 140 may be provided in a kit with or without the adjustment mechanism 108. Users may select a specific bracket 140 from among various brackets based on the angle so that the second flange 144 and the adjustment mechanism 108 will not interfere with other structures connected to the socket 110 and the pylon 112. Exemplar angles 143 between the first flange 142 and the second flange 144 includes: 60°, 70°, 80°, 90°, 100°, and 110°.

As noted above, and as shown in greater detail in FIG. 2F, the adjustment mechanism 108 includes the receiver 108a that is configured to receive the winding portion 108c of the adjustment mechanism 108. The winding portion 108c includes a knob or knob assembly 160, a spring assembly (not shown), and a spool assembly (not shown). The spool assembly includes a spool around which a portion of the cable 130 may be wound. The spring assembly includes a spring, such as a torsional coil spring, having one end in engagement with the spool. The spool assembly and the spring assembly are generally configured to be assembled to one another and placed within a housing 162 (FIG. 2F). The knob assembly 160 can then be assembled (coupled) with the housing 162 to form the winding portion 108c of the adjustment mechanism 108. The winding portion 108c may be coupled to the receiving portion 108a in any manner, such as a snap fit or with fasteners.

The knob assembly 160 and the spool assembly may be coupled together via a drive shaft (not shown) and by a gear train (not shown) as described in U.S. Pat. No. 7,992,261, which is incorporated herein by reference in its entirety. The gear train (not shown) may be provided between the knob assembly 160 and the spool assembly in order to allow a user to apply a torsional force to the winding spool that is greater than the force applied to the knob. For example, as described in U.S. Pat. No. 7,992,261, such a gear train may be in the form of an epicyclic gear set including a sun gear secured to the drive shaft and a plurality of planetary gears attached to the spool, and a ring gear on an internal surface of the housing 162. Such an epicyclic gear train will cause a clockwise rotation of the drive shaft relative to the housing 162 to result in a clockwise rotation of the spool relative to the housing 162, but at a much slower rate, and with a greater torque than that input by the user turning the knob 160. This provides a user with a substantial mechanical advantage in tightening the cable 130 using the adjustment mechanism 108. In one embodiment, the epicyclic gear train provides a gear ratio of 1:4. In alternative embodiments, other ratios can also be used as desired. For example, gear ratios of anywhere from 1:1 to 1:5 or more could be used as described therein.

The housing 162 may have an upper section with a plurality of ratchet teeth 162a configured to selectively engage pawls (not shown) on an interior side of the knob 160. Thus, in one configuration, when a user wishes to wind the spool to tighten the cable 130, a user may push the knob 160 into the housing 162 (to the left in FIG. 2F) to engage the pawls with the ratchet teeth 162a so that the user can rotate the knob 160 to rotate the spool and wind the cable 160 thereabout. The pawls prevent the coil spring from unwinding the spool as the user winds or after the user stops winding. The pawls also permit the user to unwind the spool by disengaging the pawls from the ratchet teeth, such as by pulling the knob 160 away from the housing 162, as is shown in FIG. 2F.

FIGS. 3A to 3C illustrate details of some of the anterior cable guides 131. The cable guides 131 are generally circular disc shaped and define a hole 131a in the center to receive the fastener 132 for attaching the guide 131 to the interface 120. The cable guide has a plurality of protrusions 131b, which define a curved cable path 131c for routing the cable 130, as shown in FIGS. 3A and 3B. An axis A-A bisects the cable guide 131. As noted above, to optimize the pulling forces in the cable 130 the pull angle P between the portions A and B of the cable 130 routed through the cable guide 131 is preferably bisected by the axis A-A. This can be accomplished by rotating the cable guide 131 relative to the fastener 132 and then tightening the fastener 132 to fix the position of the cable guide 131 relative to the outer surface 110c of the socket 110.

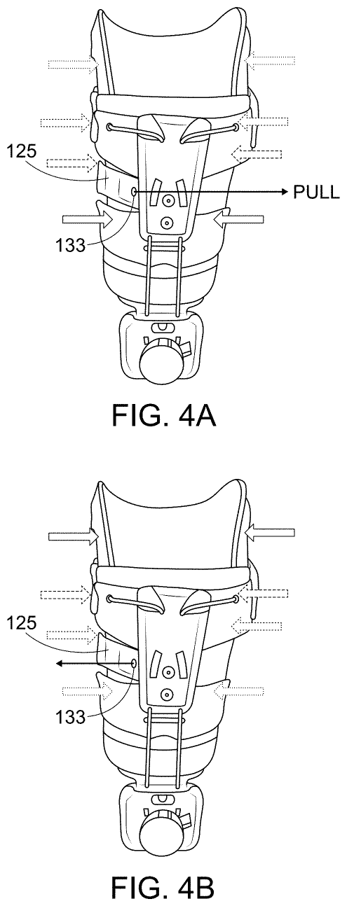

FIGS. 4A and 4B show details of a pressure adjustment using the pressure management strap 125. As shown in FIG. 4A, the strap 125 can be moved to a more lateral position by loosening the screw 133 and pulling on the strap 125 in direction of arrow labeled “PULL”, so that the socket pressure distribution will taper concentrating at the level of the patellar. Once the strap 125 is moved the screw 133 can be tightened to retain the position of the strap 125. In FIG. 4B, the strap 125 can be moved to a more medial position so that the pressure distribution will taper concentrating at the level of the medial tibial plateau.

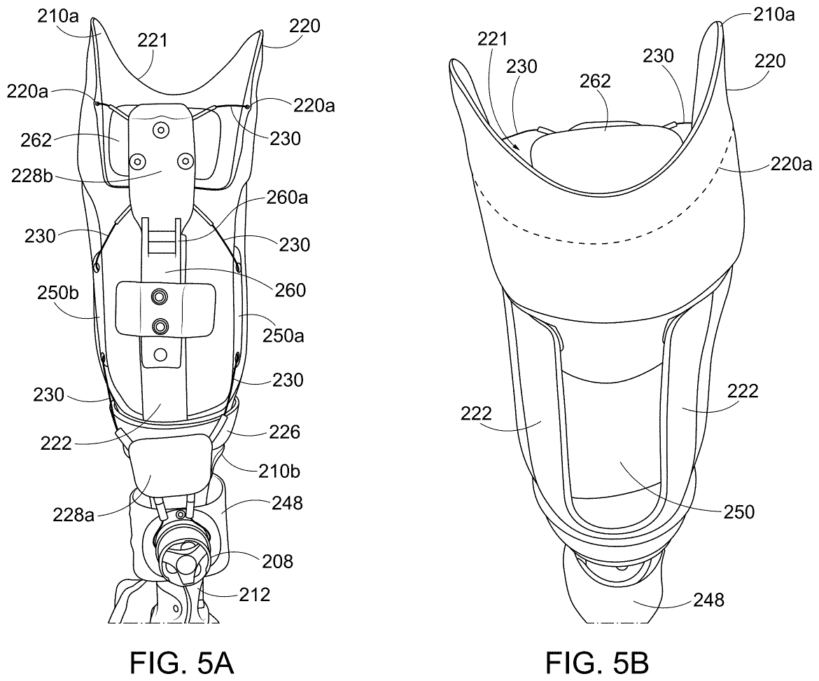

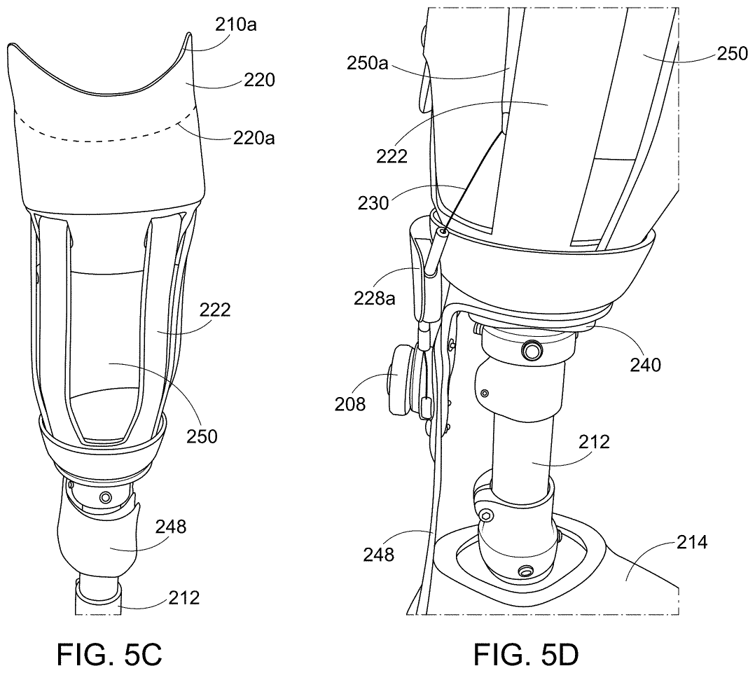

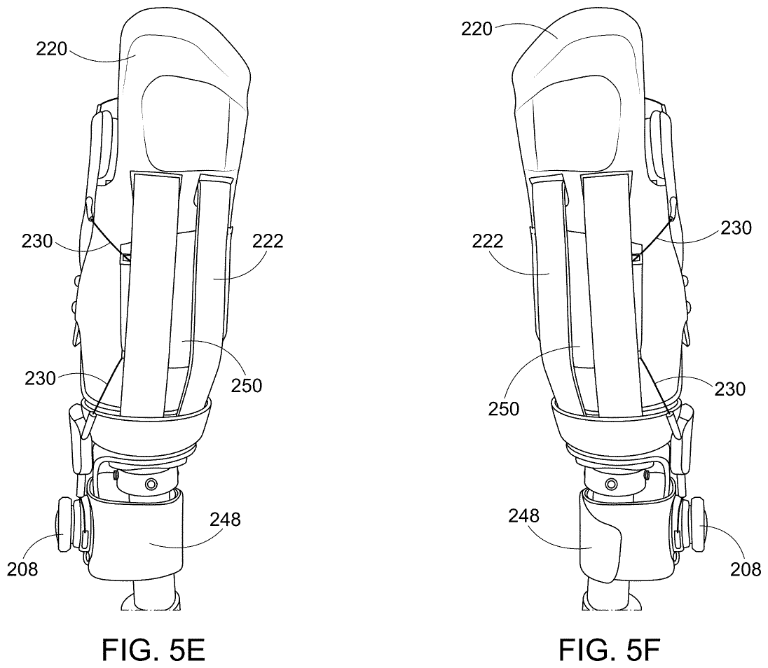

FIGS. 5A to 5F illustrate details of another embodiment of a prosthetic system 200, which is similar to system 100. In FIGS. 5A to 5F, elements of system 200 that are like those of system 100 are referred to in FIGS. 5A to 5F with reference numbers that are incremented by “100”. As shown in FIGS. 5A and 5B, a central guide member 250 is interposed between the interface 220 and the struts 222. The central guide member 250 wraps around the anterior, lateral, and medial sides of the interface 220 to circumferentially spaced ends 250a and 250b, which may be diametrically opposed. The ends 250a and 250b define elongated or tubular lumens through which the cable 230 passes in a vertical direction, as shown clearly in FIG. 5A. The lumens defined by the ends 250a and 250b may extend parallel with the struts 222 and may be adjacent thereto so that when the cable 230 is tensioned the force from the cable can be transmitted to the interface to compress the interface radially inwardly toward the cavity 221. The ends 250a and 250b of the central guide member 250 maintain lateral separation between the lateral and medial portions of the cable 230 so that those portions remain spread apart when the cable 230 is tensioned. Further, when the cable 230 is tensioned, the central guide member 250 is configured to apply radial pressure to the portion of the interface 220 in engagement with the guide member 250 due to the forces applied by the cable 230 to the guide member at the ends 250a and 250b which tend to draw the ends 250a and 250b towards one another. The central guide member 250 may be a precontoured member or a conformable member that flexibly extends through its interpositional routing. The central guide member 250 is longitudinally (circumferentially) inelastic. It may be made of various materials, including textiles and/or polymers.

Also shown in FIG. 5A is a lower cable guide 228a that is attached to a distal end 210b of the socket 210 and spaced vertically above the adjustment mechanism 208. The lower cable guide 228a guides the cable 230 from the adjustment mechanism 208 through the lower cable guide 228a upward to the central guide member 250. The adjustment mechanism 208 is connected to the pylon 212 via an adjustable strap 248.

An upper cable guide 228b is attached to one of the struts 222 via an arm 260 and is preferably aligned vertically with the lower cable guide 228a. The upper cable guide 228b may be pivotally connected to the arm 260 with a hinge 260a or may be disposed on a resiliently flexible portion of arm 260 to permit the upper cable guide 228b to be displaced radially in and out of the cavity 221. The upper cable guide 228b is connected to a pad 262 that moves with the upper cable guide 228b in and out of the cavity based on the tension in the cable 230. The pad 262 is configured to engage a residual limb in the cavity 221. The upper cable guide 228b guides the cable 230 between the proximal end of the central guide member 250 and a proximal channel 220a in the interface 220. As shown in FIG. 5B, the cable 230 extends circumferentially around the interface 220 in the proximal channel 220a.

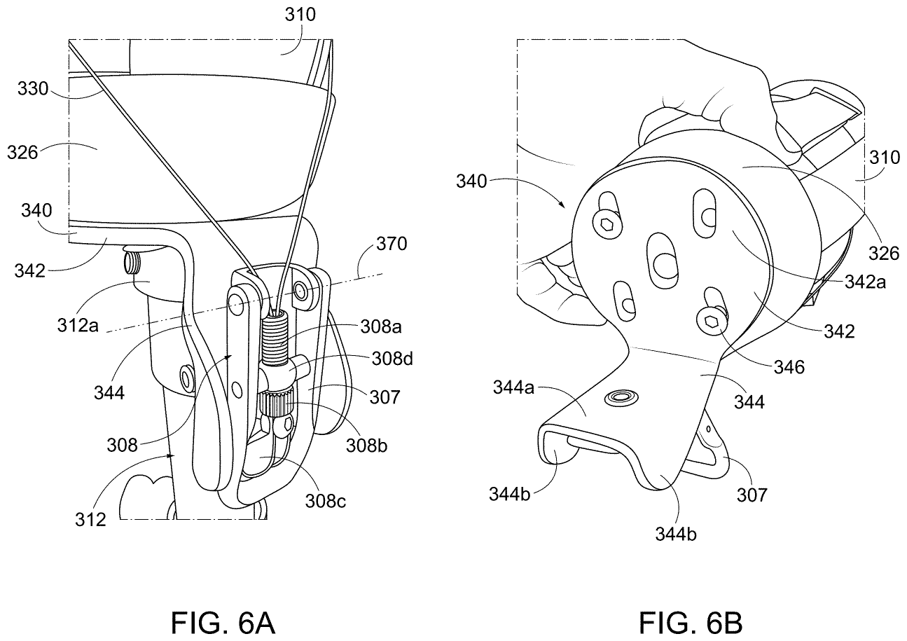

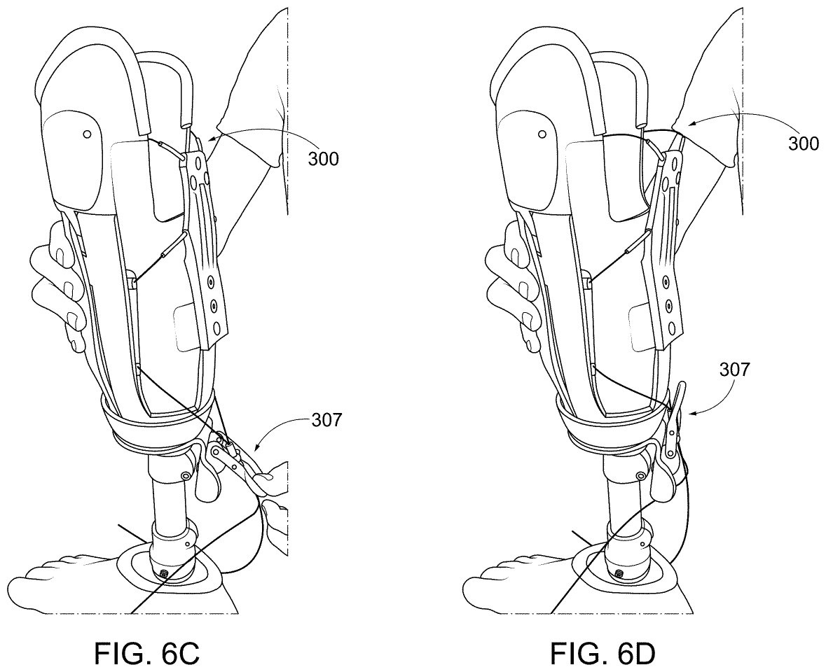

FIGS. 6A to 6D show features of another embodiment of a prosthesis system 300. In FIGS. 6A to 6D like elements to those of system 100 are referred to in FIGS. 6A to 6D incremented by “200”. Thus, FIGS. 6A and 6B show an alternative adjustment mechanism 308 and a bracket 340. The adjustment mechanism 308 is coupled to a latch lever 307, which is pivotally coupled to the bracket 340 to pivot in a vertical plane. The cable adjuster 308 operatively moves with the lever 307 when the lever 307 is rotated between open and closed positions, further details of which are described below.

In FIG. 6A the lever 307 is shown in a closed position. The cable 330 is routed about the socket 310 and two portions of the cable 330 are secured to each other at a retainer 308c, which may be a crimped connection or other fastener means. In FIG. 6A, the retainer 308c and the retained portions of the cable 330 are pulled down to tighten a routed portion of the cable 330 about the socket 310. In FIG. 6, the lever 307 is shown in an open position in which the retainer 308c and the retained portions of the cable 330 are raised to loosen the routed portion of the cable 330 about the socket 310. In FIG. 6C, the lever 307 is shown in an intermediate configuration between the open and closed configuration.

The cable adjuster 308 includes an externally threaded adjustment screw 308a having a head 308b (which can be turned by hand by a user during a setup or an adjustment operation, preferably when the lever 307 is in the open position), the cable retainer 308c, and an adjustment nut 308d which is threaded with the adjustment screw 308a. The portion of the cable 330 between the two retained portions of the cable 330 forms a loop that is routed about the socket 310. As shown in FIG. 6A, the screw head 308b is configured to engage and bear against the cable retainer 308c so that when the lever 307 is in the closed position, the screw head 308b pushes down on the cable retainer 308c to pull on the looped ends of cable 330 routed about the socket 310, thus tightening the cable 330 about the interface 320.

In the embodiment shown in FIGS. 6A to 6D, the cable 330 extends through an inner lumen of the screw 308a to the cable retainer 308c. The screw 308a extends through the nut 308d, which is secured to the lever 307. The adjustment screw 308a can be threaded with respect to nut 308d to adjust the position of the head 308b and, thus, the cable retainer 308c relative to the nut 308d. This, in turn, adjusts the position of the looped ends of the cable 330 relative to the nut 308d, which will affect the amount of tension in the cable 330 when the lever 307 is closed. Once the adjustment screw 308a is adjusted and set in the nut 308d, the length of the portion of the cable 330 that is laced around the socket 310 in the closed position of the lever 307 remains fixed until the adjustment screw 308a is readjusted to a different setting.

As shown in FIG. 6A, when the lever 307 is rotated about pivot axis 370 down into the closed and “overcenter” position (i.e., such that the force vector of the cable tension between the screw 308a and the lower cable guide 228a extends between the pivot axis 370 and the second flange 344), the retained portions of the cable 330 at the retainer 308c are pulled downwardly the farthest from the interface 320, and locked in tension. Thus, the routed portion of the cable 330 about the socket 310 is at its tightest position. When the lever 307 is rotated upwardly, away from the overcenter position, as in FIGS. 6C (intermediate position) and 6D (open position), the retained portions of the cable 330 at the retainer 308c are raised upwardly with the lever 307 closer to the interface 320, thereby relieving tension in the portion of the cable 330 that is routed around the socket 310, loosening the connection between the socket 310 and any residual limb therein. Thus, the adjuster 308 provides a fine adjustment of the length of cable 330 laced about the socket 310, and the latch lever 307 applies the tension in a discrete (on/off) manner. Once the user closes the lever 307 the tension in the cable 330 is not readily changed, until the user opens the lever 307, adjusts the nut 308b, and then closes the lever 307.

The bracket 340 has a first portion 342 and a second portion 344 that extends downward at a non-zero angle relative to the first portion 342. The first portion 342 resembles the configuration of first portion 142 in that the first portion 342 includes elongated holes 342a that are arranged in a pattern to align with one or more of holes in base 326 of socket 310 to receive fasteners 346. While FIG. 6B shows fasteners 346 connecting the first portion 342 of the bracket 340 directly to the base 326, in more common practice, the first portion 342 will be interposed between the flange 312a of pylon 312 and the base 326, as shown in FIG. 6A and described herein. The second portion 344 has a central portion 344a to which is mounted the adjustment mechanism 308. The second portion 344 also includes side wings 344b which can act to guard the handle 308b from the sides when the lever 307 is in the closed configuration shown in FIG. 6A.

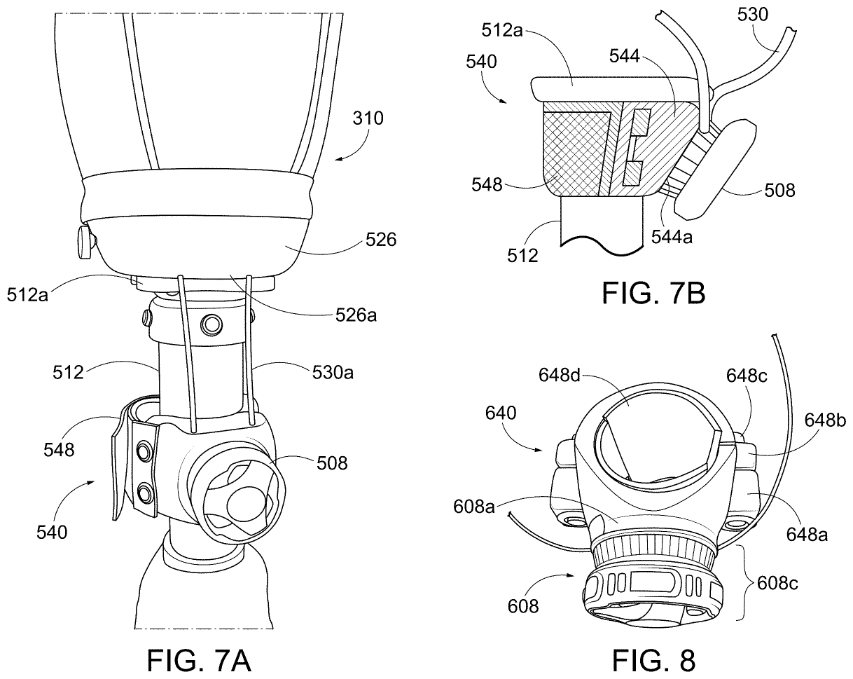

FIGS. 7A, 7B, and 8 show other embodiments of mounting arrangements for respective adjustment mechanisms. In FIGS. 7A and 7B, elements that are like those of system 100 are referred to in FIGS. 7A to 7 with reference numbers that are incremented by “400”.

FIG. 7A shows a mounting arrangement 540 for an adjustment mechanism 508. As shown in FIG. 7B, the mounting arrangement 540 includes a body or housing 544 with a receiver 544a to receive the adjustment mechanism 508 and an adjustable strap 548 extending from the body or housing. The strap 548 is configured to securely band about a pylon 512 so that the adjustment mechanism 508 is secured to the pylon 512. As shown in FIG. 5A, cable housing 530a is routed through holes 526a in a base 526 of a socket 510. The holes 526a in the base 526 are located beyond a proximal flange 512a of the pylon 512 so that the flange 512a does not interfere with the cable housings 530a. In FIG. 7A the adjustment mechanism is spaced vertically from the flange 512a, whereas in FIG. 7B, the adjustment mechanism is located adjacent the pylon's proximal flange 512a. In either position in FIGS. 7A or 7B, the strap 548 provides some amount of flexibility to position the adjustment mechanism such that the cable in cable housings 530a enters the adjustment mechanism 508 without tight bends.

FIG. 8 shows a mounting arrangement 640 for an adjustment mechanism 608. In FIG. 8, elements that are like those of system 100 are referred to in FIG. 8 with reference numbers that are incremented by “500”. The mounting arrangement includes a clamp 648, which is configured to securely fasten to a pylon, such as pylon 512 of FIG. 7A. The clamp 648 shown has a first portion 648a and a second portion 648b, which are coupled together with fasteners 648c (e.g., threaded fasteners). The first and second portions 648a, 648b can be completely separated from one another to open the clamp 648 to position the portions 648a, 648b around a pylon (e.g., pylon 512) prior to fastening the portions 648a, 648b together about the pylon with the fasteners 648c. The clamp 648 also has a high friction material 648d, such as rubber, on the inside of the first and second portions 648a, 648b. When the portions 648a, 648b of the clamp 648 are tightly fastened together about the pylon, the high friction material grips the pylon and limits movement of the clamp 648 with respect to the pylon.

The first portion 648a of the clamp 648 is connected to a receiver 608a, which may be identical to the receiver 108a discussed above for receiving a winding portion 608c, which may be same as the winding portion 108c, also discussed above. The receiver 608a can be attached to or integrally formed (i.e., molded into) with the first portion 648a.

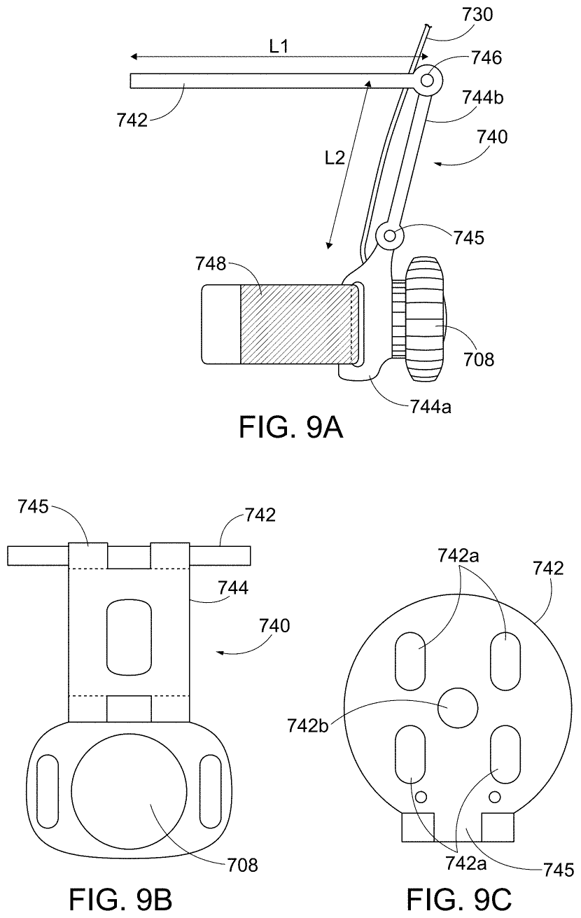

FIGS. 9A to 9C show another embodiment of a mounting arrangement 740 for an adjustment mechanism 708. In FIG. 9A, an adjustment mechanism 708, which can be the same as adjustment mechanism 108, is mounted to a bracket 744a. The bracket 744a defines holes or slots through which an adjustable strap 748 is coupled for attaching the bracket 744a to a pylon, such as pylon 512. The bracket 744a is pivotally coupled at pivot 745 to a linkage 744b, which is pivotally coupled at a pivot 746 to a mounting flange 742. As shown in FIG. 9C, the mounting flange 742 defines a plurality of elongated holes 742a in a pattern around a central hole 742b.

The adjustment mechanism 708 can be mounted to a socket and a pylon, such as socket 110 and pylon 112, in similar fashion described above in connection with FIGS. 2A to 2C. In that regard, the holes 742a and 742b can be aligned with the fasteners 146 in the same way as the holes 142a of flange 142, to connect the flange 742 between the base 126 and the flange 112a of the pylon 112. Once the flange 742 is connected between the base 126 and the flange 112a, the strap 748 can be securely fastened around the pylon.

It will be appreciated that the lengths L₁ and L₂ can be varied in various arrangements 740 to provide more or less clearance in the area between the mounting flange 742 and the linkage 744b. Multiple arrangements 740 with varying lengths L₁ and L₂ may be provided as a kit with or without the adjustment mechanism 708 so that a user can select a suitable arrangement 740 that will not interfere with other structures when the user connects the arrangement to the user's specific socket and prosthetic extremity.

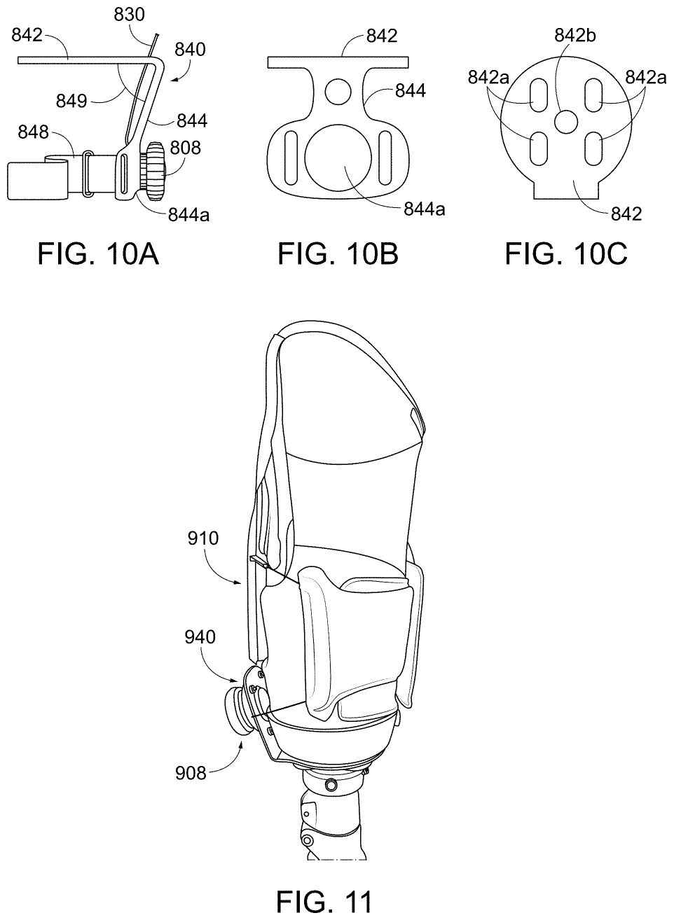

FIGS. 10A to 10C show another embodiment of a mounting arrangement 840 in the form of a bracket for an adjustment mechanism 808. In FIG. 10A, the adjustment mechanism 808, which can be the same as adjustment mechanism 108, is mounted to a lower flange 844 having a receiver 844a and fixedly coupled to a mounting flange 842 that extends at an angle 849, which is fixed. Various brackets 840 can be made with different preformed angles between the mounting flange 842 and the lower flange 844 so that users can select a bracket 840 that will not interfere with any other components of the socket or pylon. The lower flange 844 is coupled to a strap 848 for securely fastening the lower flange 844 of the bracket 840 to a pylon, such as pylon 112. Also, as shown in FIG. 10C, the mounting flange 842 defines a plurality of elongated holes 842a in a pattern around a central hole 842b.

The adjustment mechanism 808 can be mounted to a pylon, such as pylon 112, in similar fashion described above in connection with FIGS. 2A to 2C. In that regard, the holes 842a and 842b can be aligned with the fasteners 146 in the same way as the holes 142a of flange 142, to connect the mounting flange 842 between the base 126 and the flange 112a of the pylon 112. Once the mounting flange 842 is connected between the base 126 and the flange 112a, the strap 848 can be securely fastened around the pylon.

While the foregoing embodiments have shown the various adjustment mechanisms disposed below the socket, it will be appreciated that the foregoing mounting arrangements can also be used to dispose the adjustment mechanism above the distal end of the socket. For example, FIG. 11 shows an embodiment of an arrangement where a bracket 940, which may be the same as bracket 140, is inverted and used to dispose an adjustment mechanism 908 above the distal end 110b of the socket 910, which may not resolve the disadvantages noted about the location of the adjustment mechanism from the prior art, but may provide an easier mechanism for such assembly.

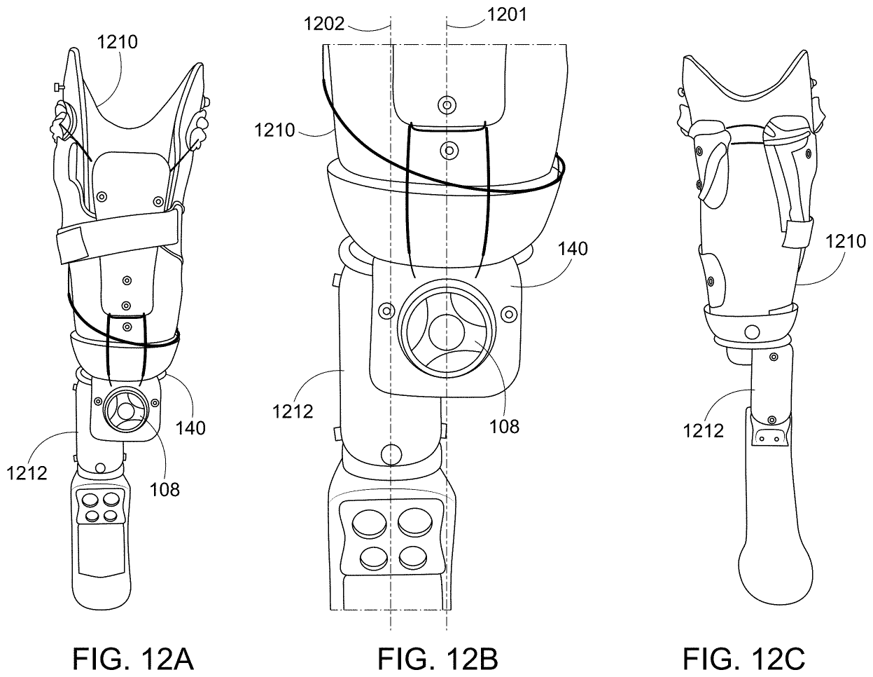

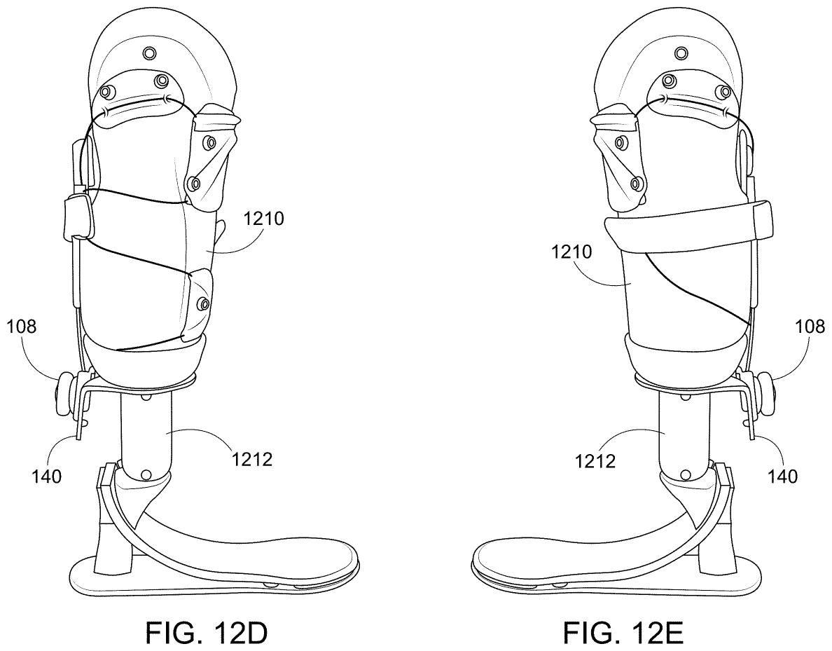

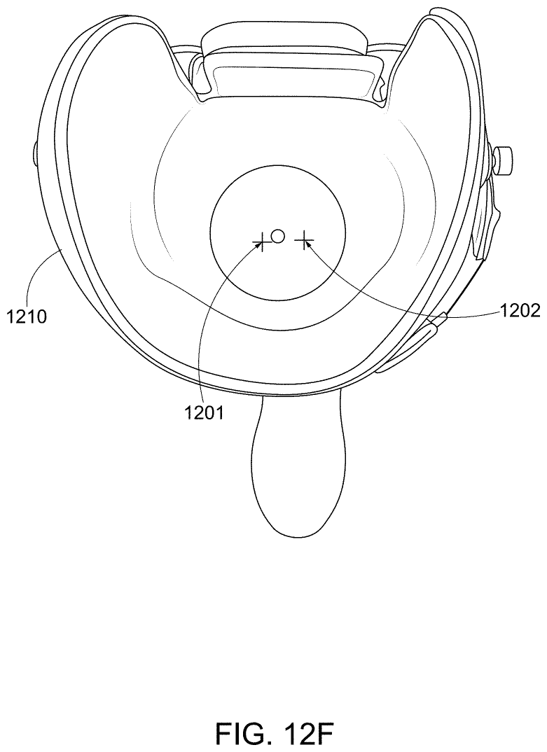

As noted above, the various brackets 140, 340, 740, 840, have respective flanges 142, 342, 742, 842 with elongated holes arranged in a pattern that permit a range of axial misalignment between a socket (e.g., socket 100) and a depending portion (e.g., a flange 112a of pylon 112). FIGS. 12A to 12E shows an example of a prosthetic system 1200 in accordance with the disclosure. In FIGS. 12A to 12E elements corresponding to those of system 100 are incremented by “1100”. The bracket 140 and adjustment mechanism 108 are connected to the pylon 1212 and the socket 1210. However, the pylon 1212 is axially misaligned with the socket 1210. Specifically, as shown in FIGS. 12A to 12C, the axis 1202 of the pylon 1212 is offset in the medial direction with respect to the axis 1201 of the socket 1210.

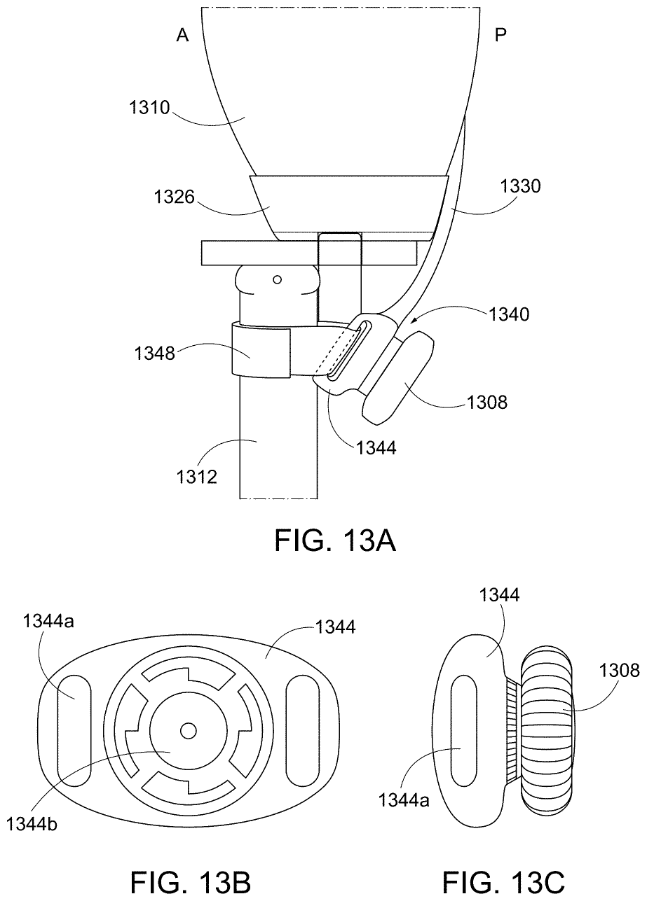

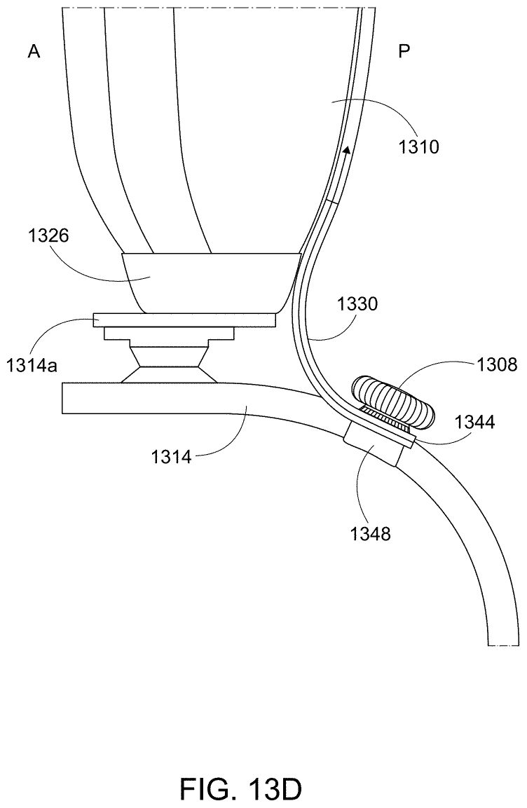

FIGS. 13A to 13D show another embodiment of a mounting arrangement 1340 for an adjustment mechanism 1308. In FIGS. 13A to 13E elements corresponding to those of system 100 are incremented by “1200”. In FIG. 13A the socket 1310 is positioned axially offset in the posterior direction with respect to the pylon 1312. Similarly, in FIG. 13D, the socket 1310 is positioned axially offset in the posterior direction with respect to a flange 1314a of the foot-ankle structure 1314. The mounting arrangement 1340 is similar to the mounting arrangement 840, but does not include the mounting flange 842 and has a modified version of the bracket flange 844. Specifically, the mounting arrangement 1340 includes a bracket or flange 1344 (shown in detail in FIGS. 13C and 13D) that receives and supports the adjustment mechanism 1308, which can be the same as adjustment mechanism 108. The flange 1344 defines a cavity 1344b (FIG. 13B), which is configured to receive a winding portion of the adjustment mechanism 1308, which may be the same as the winding portion 108c (FIG. 2A) of the adjustment mechanism 108. The bracket or flange 1344 may have openings 1344a for connection to a strap 1348, which can be banded about various structures, including a pylon 1312 (FIG. 13A) and a portion of an ankle-foot structure 1314 (FIG. 13C). As shown in FIG. 13A, when the bracket 1344 is coupled to the pylon 1312 with the strap 1348, cables 1330 from the adjustment mechanism 1308 bend around the base 1326 of the socket 1310, which are axially misaligned in FIG. 13A. Also shown in FIG. 13B, the cables 1330 can bend around the base 1326 of the socket 1310v even when the flange of the angle foot structure 1314 is axially misaligned with the socket 1310.

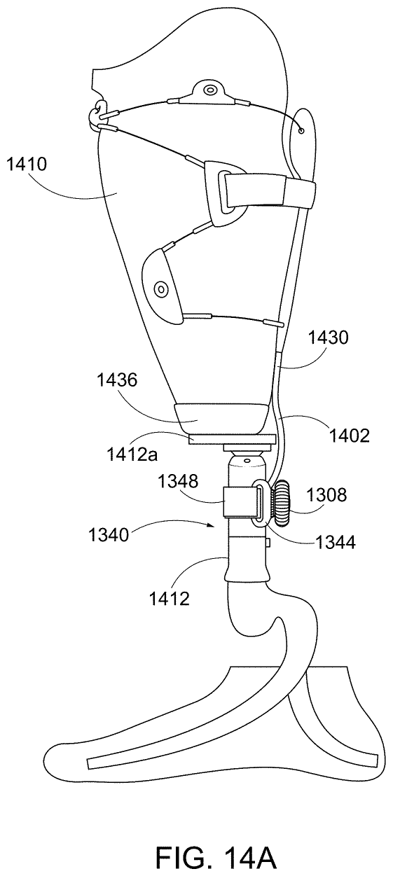

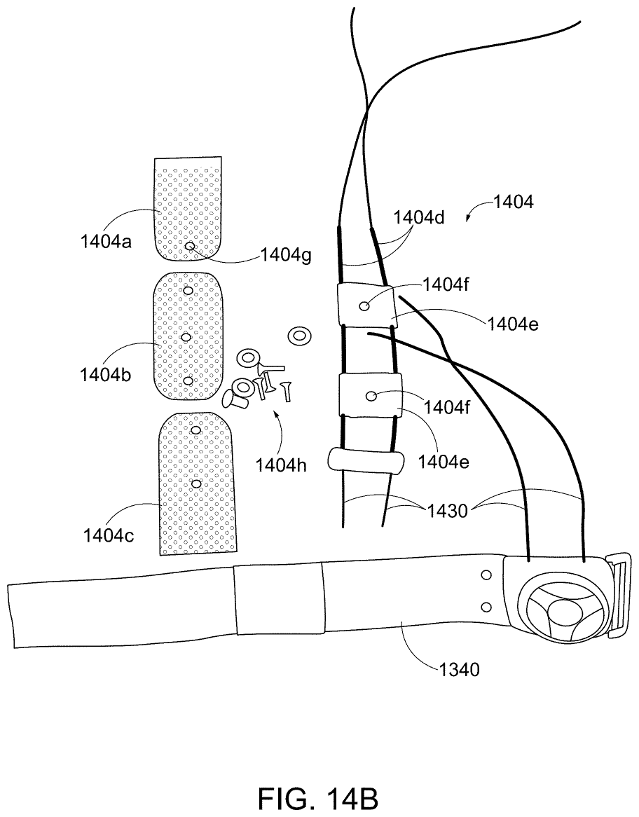

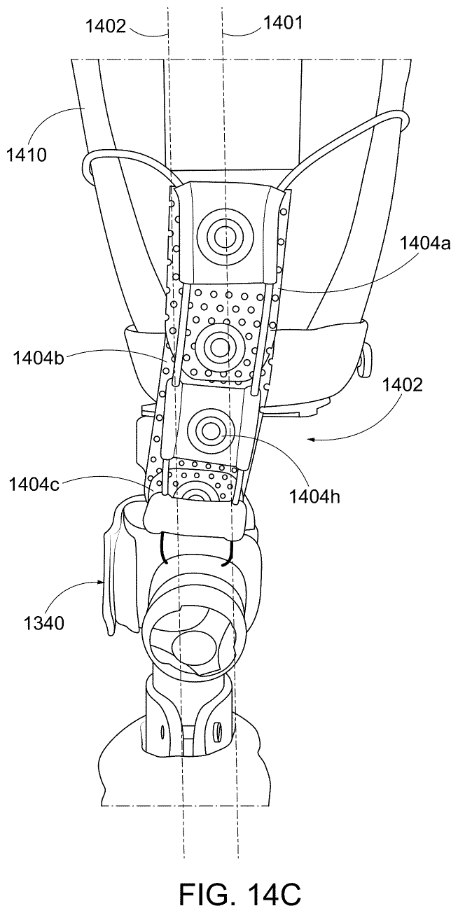

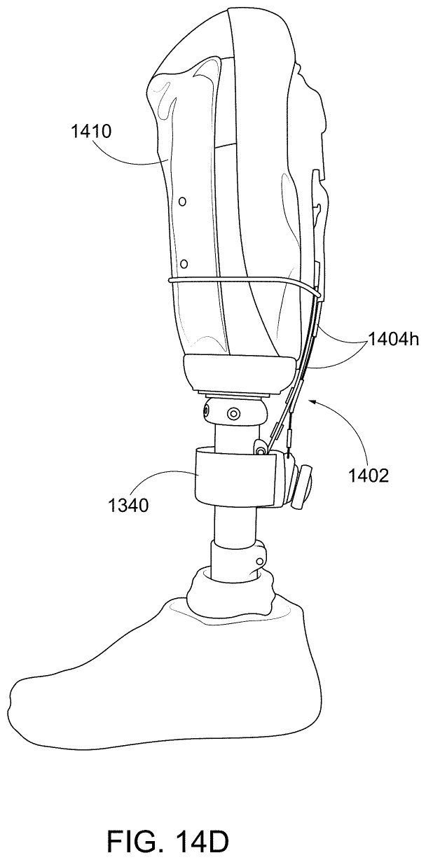

To provide additional mechanical support for the cables 1330, the mounting arrangement 1340 may be combined with a cable guide 1402 extending from the socket 1410 to the bracket 1344, as shown in FIG. 14A. Specifically, the cable guide 1402 is a flexible member that can curve around the base 1436 and pylon flange 1412a and any other obstruction to route cables 1430 from the adjustment mechanism 1308 to the socket 1410. FIG. 14B shows components of a kit 1404, which comprises the cable guide 1402.

The kit 1404 includes a plurality of flexible, planar segments or links 1404a, 1404b, and 1404c, cable housings 1404d that house cables 1430, and cable housing spacers 1404e that laterally space the cable housings 1404d from each other. The cable housing spacers 1404e define through holes 1404f, which are configured to align with through holes 1404g in segments or links 1404a, 1404b, and 1404c. Screw fasteners 1404h are inserted into the aligned through holes to join the segments or links 1404a, 1404b, and 1404c, and some of such screw fasteners 1404h may also secure to mating fasteners on the socket 1410, to thereby secure at least one portion of the cable guide 1402 to the socket 1410, in as shown in FIGS. 14C and 14D. Thus, the fasteners 1404h can form joints between the segments 1404a, 1404b, and 1404c so that the segments can be pivoted relative to one another so that the assembled cable guide 1402 can be set to extend along a curve.

For example, as shown in FIG. 14C, a vertical axis 1401 of the socket 1410 is laterally offset from a vertical axis 1402 of the pylon 1412. Thus, when the mounting arrangement 1340 and the adjustment mechanism 1308 are connected to the pylon 1412, they will also be laterally offset from the socket 1410. To support the cable housings 1404d, the segments 1404a, 1404b, and 1404c are connected to one another and to the cable housings 1404d via the cable housing spacers 1404e and the fasteners 1404h. The segments 1404a, 1404b, and 1404c can be pivoted relative to one another about the fasteners 1404h to align with the curvature of the cable housings 1404d and match the lateral offset. In the example shown in FIGS. 14C and 14D, the upper two fasteners 1404h are also secured to the socket 1410.

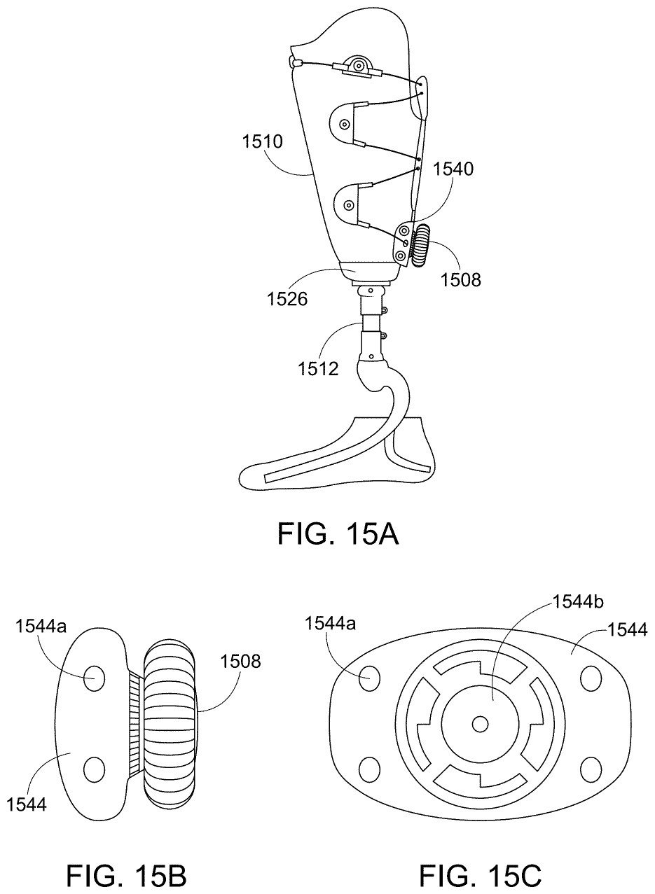

FIGS. 15A to 15C relate to yet another embodiment of a mounting arrangement 1540 for mounting an adjustment mechanism at or above the base 1526 of the socket 1510. In FIGS. 15A to 15C elements corresponding to those of system 100 are referred to with reference numbers incremented by “1400”. Specifically, the mounting arrangement 1540 includes a bracket or flange 1544, shown in greater detail in FIGS. 15B and 15C, that receives and supports the adjustment mechanism 1508, which can be the same as adjustment mechanism 108. The bracket or flange 1544 has openings 1544a to receive a fastener, such as a screw, which can be received by a mating fastener affixed to the socket 1510, as shown in FIG. 15A. Thus, the mounting arrangement 1540 does not require any connection between the base 1526 and the structure below it, i.e., pylon 1512. By disposing the adjustment mechanism 1508 at or above the base 1526 of the socket 1510, all possible interference between the adjustment mechanism 1508 and structures below the socket 1510 can be eliminated.

There have been described and illustrated herein several embodiments of a system and a method of use. While particular embodiments of the invention have been described, it is not intended that the invention be limited thereto, as it is intended that the invention be as broad in scope as the art will allow and that the specification be read likewise. Thus, while particular embodiments of adjustment mechanisms have been disclosed, it will be appreciated that other mechanisms may be used as well. Also, while a cable has been disclosed for application of tension to adjust the size of the cavity in the socket, other tension element including, not by way of limitation, wires, filamentary and multifilamentary structures may be used as well. The cable may preferably be inelastic, although in at least one embodiment, the cable may have the ability to elastically deform a minimum amount. In addition, while particular types of adjustment mechanism mounts have been disclosed, it will be understood that other connections to dispose the adjustment mechanism below the socket can be used. It will therefore be appreciated by those skilled in the art that yet other modifications could be made to the provided invention without deviating from its spirit and scope as claimed.

What is claimed is:

1. A prosthesis system for connection to a user's residual limb, the system comprising:
a socket having an open proximal end and a closed distal end and defining a cavity to receive the residual limb;
a tension element extending about a portion of the socket, the tension element having at least one portion extending below a distal end of the socket;
an adjustment mechanism coupled to the socket and disposed below the distal end of the socket, the adjustment mechanism coupled to the at least one portion of the tension element extending below the distal end of the socket, the adjustment mechanism configured to adjust the tension in the tension element such that a portion of the socket is displaced in relation to another portion to adjust the fit between the socket and the residual limb; and
a prosthetic extremity coupled to the socket and extending below the socket.

2. The system according to claim 1, further comprising at least one of:
i) a mounting bracket extending from the adjustment mechanism to an interface between the distal end of the socket and the prosthetic extremity;
ii) a strap extending from the bracket or the adjustment mechanism to the prosthetic extremity; and
iii) a clamp attached to the adjustment mechanism, the clamp attaching the adjustment mechanism to the prosthetic extremity.

3. The system according to claim 2, wherein the system includes the mounting bracket and the bracket has a first flange that defines at least one hole, the at least one hole configured to align with a corresponding fastener between the distal end of the socket and the prosthetic extremity, and the bracket has a second flange extending at an angle with respect to first flange, the adjustment mechanism mounted to the second flange, the second flange preferably extending downward at an angle relative to the first flange.

4. The system according to claim 1, wherein the socket defines pathways through which the tension element is routed.

5. The system according to claim 4, wherein the pathways include channels.

6. The system according to claim 1, further comprising guides coupled to the socket, the guides configured to guide the tension element about the socket.

7. The system according to claim 1, wherein the adjustment mechanism is configured to provide variable tension adjustment to the tension element.

8. The system according to claim 7, wherein the adjustment mechanism is a rotary winding mechanism.

9. The system according to claim 1, wherein the adjustment mechanism is configured to tighten and loosen the tension element about the socket in preset adjustments.

10. The system according to claim 9, wherein the adjustment mechanism comprises a folding lever and an overcenter mechanism that locks the lever between an open configuration and a closed configuration.

11. The system according to claim 1, wherein the tension element extends around a circumference of the socket.

12. The system according to claim 1, wherein the socket includes a plurality of struts and an interface supported by the struts, the interface defining the cavity.

13. The system according to claim 12, wherein the tension element is routed through or along the struts, whereby the struts are configured to move radially in response to tension in the tension elements.

14. The system according to claim 1, wherein the tension element is a cable.

15. The system according to claim 1, wherein the prosthetic extremity includes a prosthetic foot and a longitudinal pylon to displace the prosthetic foot relative to the socket.

16. A prosthesis system for connection to a user's residual limb, the system comprising:
a socket having an open proximal end and a closed distal end and defining a cavity to receive the residual limb;
a tension element extending about a portion of the socket,
an adjustment mechanism coupled to the socket and the tension element, the adjustment mechanism configured to adjust the tension in the tension element such that the size of the cavity is adjusted to modify the fit between the socket and the residual limb;
a prosthetic extremity coupled to the socket and extending below the socket; and
a bracket configured to connect to at least one of the socket and the prosthetic extremity, the bracket having a receiver for receiving the adjustment mechanism so that the bracket supports and disposes the adjustment mechanism displaced from the socket.

17. A prosthesis system for connection to a user's residual limb, the system comprising:
a socket having an open proximal end and a closed distal end and defining a cavity to receive the residual limb;
a tension element extending about a portion of the socket,
an adjustment mechanism coupled to the socket and the tension element, the adjustment mechanism configured to adjust the tension in the tension element such that the size of the cavity is adjusted to modify the fit between the socket and the residual limb;
a prosthetic extremity coupled to the socket and extending below the socket; and
a strap configured to connect to the prosthetic extremity, the strap configured for banding about at least a portion of the prosthetic extremity to securely dispose the adjustment mechanism to the prosthetic extremity.

18. The system according to claim 17, wherein the prosthetic extremity includes a prosthetic foot and a longitudinal pylon to displace the prosthetic foot relative to the socket, and the strap bands about the pylon.

19. In a prosthesis system for connection to a user's residual limb, the system comprising a socket having an open proximal end and a closed distal end and defining a cavity to receive the residual limb, a tension element extending about a portion of the socket, and an adjustment mechanism coupled to the at least one portion of the tension element, the adjustment mechanism having a spool configured to wind the tension element and the adjustment mechanism configured to adjust the tension in the tension element such that the size of the cavity is adjusted to modify the fit between the socket and the residual limb, the improvement comprising:
a bracket coupled at the distal end of the socket and above the pylon and having a receiver for receiving the adjustment mechanism so that the bracket supports and disposes the adjustment mechanism relative to the socket.

20. The improvement according to claim 19, wherein the adjustment mechanism is interchangeable in the receiver with another adjustment mechanism having another spool.

21. The improvement according to claim 19, wherein the bracket disposes the adjustment mechanism displaced from the socket.

22. The improvement according to claim 19, wherein the bracket disposes the adjustment mechanism below the distal end of the socket.
